(19) United States
(12) Patent Application Publication
(10) Pub. No.: US 2026/0053875 A1
(43) Pub. Date: Feb. 26, 2026

(54) TRANSDERMAL DRUG-CARRIER VEHICLE FOR TOPICAL, LYMPHATIC, MUSCULAR, AND SYSTEMIC DELIVERY OF BIOACTIVE ANDROGRAPHOLIDES, AND/OR EPIGALLOCATECHIN GALLATE

(71) Applicant: Counterpoint Biomedica LLC, Santa Monica, CA (US)

(72) Inventors: Amonnat Srichonpaisarn Hall, Carmel Valley, CA (US); Frederick Lewis Hall, Carmel Valley, CA (US); Erlinda Maria Gordon, Santa Monica, CA (US)

(21) Appl. No.: 19/306,582

(22) Filed: Aug. 21, 2025

Related U.S. Application Data
(60) Provisional application No. 63/685,955, filed on Aug. 22, 2024.

(51) Int. Cl. A61K 36/19; A61K 9/00; A61K 9/06; A61K 36/82; A61K 47/10; A61K 47/14; A61K 47/22; A61K 47/34; A61K 47/44; A61P 19/02

(52) U.S. Cl. CPC A61K 36/19; A61K 9/0014; A61K 9/06; A61K 36/82; A61K 47/10; A61K 47/14; A61K 47/22; A61K 47/34; A61K 47/44; A61P 19/02

(57) ABSTRACT

Described herein are compositions (e.g., topical formulations) including andrographolides (ANGL), epigallocatechin gallate (EGCG), or a combination thereof, and methods of making and using the same.

### CROSS-REFERENCE TO RELATED APPLICATION

This application claims priority to U.S. Provisional Patent Application 63/685,955, filed Aug. 22, 2024, the contents of which are hereby incorporated by reference.

### TECHNICAL FIELD

Described herein are compositions and formulations for the topical or transdermal delivery of andrographolides and/or epigallocatechin gallate, and methods of making the same.

### BACKGROUND

Andrographis paniculata (Burm.f.) Nees is a well-known Asian medicinal plant of the Acanthaceae family. The aerial parts, roots and whole plant of A. paniculata have been used for centuries in Asia as traditional medicine for the treatment of various ailments. Several studies showed that this plant exhibited various biological activities such as anti-microbial, cytotoxicity, anti-protozoan, anti-inflammatory, anti-oxidant, immunostimulant, anti-diabetic, anti-infective, anti-angiogenic, hepato-renal protective, sex hormone modulatory, liver enzymes modulatory, vascular protective, insecticidal and toxicity activities.

A. paniculata has various compounds in its aerial parts and roots and these are often used in extracting its active principles. Diverse factors such as geographical region, harvest time and processing method account for the variability in its chemical content. Phytochemical studies of A. paniculata has led to the isolation of various bioactive plant metabolites. Notable among these metabolites are the ent-labdane diterpene lactones which account for a large proportion of its components and therapeutic activity. “Andrographolides” or ANGL is the term that is used to refer to the diterpene lactone compounds extracted from A. paniculata. Other categories of compounds that have also been isolated include flavonoids (flavones), terpenes, xanthones, polyphenols, and trace and macro elements.

Despite its wide-ranging therapeutic characteristics, extracted Andrographolides have low bioavailability and poor water solubility, which can limit biodistribution and accumulation in the body after administration. In addition, Andrographolide is not stable in gastrointestinal alkaline and acidic environments and has been reported to have a very short half-life.

Epigallocatechin gallate (EGCG), also known as epigallocatechin-3-gallate, is the ester of epigallocatechin and gallic acid, and is a type of catechin. It's known for its potential benefits, including anti-inflammatory, anti-cancer, and heart-healthy properties. EGCG may also contribute to weight loss and brain protection. When taken orally, EGCG has very poor absorption, even at daily intake equivalent to 8 to 16 cups of green tea, an amount causing adverse effects such as nausea or heartburn. The degrading of EGCG to lesser active catechins, upon oral consumption and resulting first pass metabolism, results in exceedingly low bioavailability, thus precluding potential therapeutic blood levels.

To advance the therapeutic utility of these potent and selective, yet chemically fragile biomolecules, there is a need in the art to provide alternative modes, methods, compositions, and/or formulations comprising purified bioactive andrographolides and/or epigallocatechin gallate that exhibit improved delivery and enhanced bioavailability.

### SUMMARY

Provided herein are topical formulations comprising: a) Andrographolides (ANGL) (i.e., Andrographis paniculata, full spectrum extract), Epigallocatechin Gallate (EGCG), or a combination thereof, b) a wetting/hydrating agent; c) a vegetable oil; d) a natural triglyceride emollient; e) a polymeric emulsifier/rheological modifier; f) a silicone oil; g) a natural antioxidant; h) a lipid preservative; and i) an antimicrobial/cosmetic preservative.

In some embodiments, the formulation comprises bioactive andrographolides, ANGL; and ANGL is a powdered extract of Andrographis paniculata (leaf) that is standardized to ≥50% ANGL.

In some embodiments, the ANGL is present in an amount of at least 0.5% or more based on the total weight of the formulation.

In some embodiments, the ANGL is present in an amount of at least 1.0% or more based on the total weight of the formulation.

In some embodiments, the ANGL is present in an amount of at least 1.5% or more based on the total weight of the formulation.

In some embodiments, the ANGL is present in an amount of at least 2.0% or more based on the total weight of the formulation.

In some embodiments, the formulation comprises a combination of EGCG and ANGL, wherein EGCG is a powdered extract of C. sinensis (leaf) that is ≥94% pure, and ANGL is a full spectrum extract that is >50% pure. In some instances, the ANGL is present in an amount of at least 0.5% or more based on the total weight of the formulation. In some instances, the EGCG is present in an amount of at least 1.0% or more based on the total weight of the formulation.

In some embodiments, the formulation comprises a combination of EGCG and ALA, wherein is a powdered extract of C. sinensis (leaf) that is ≥94% pure. In some instances, the EGCG is present in an amount of at least 1.0% or more based on the total weight of the formulation and the ALA is present in an amount of at least 0.6% or more based on the total weight of the formulation.

In some embodiments, the wetting/hydrating agent is glycerin, and wherein the wetting/hydrating agent further comprises distilled water. In some instances, the glycerin is present in an amount of at least about 6.0% or more based on the total weight of the formulation. In some instances, the glycerin is present in an amount of at least about 7.0% or more based on the total weight of the formulation.

In some embodiments, the vegetable oil is Avocado Oil, Macadamia Nut Oil, or Olive Oil. In some instances, the vegetable oil is present in an amount of at least 3.0% or more based on the total weight of the formulation.

In some embodiments, the natural triglyceride emollient is Caprylic/Capric triglyceride. In some instances, the Caprylic/Capric triglyceride is present in an amount of at least 4.0% or more based on the total weight of the formulation.

In some embodiments, the polymeric emulsifier/rheological modifier is Sodium Acrylate/Sodium Acryloyldimethyl Taurate Copolymer-C15-19 Alkane-Polyglyceryl-6 Laurate-Polyglycerin-6. In some instances, the polymeric emulsifier/rheological modifier is present in an amount of at least 3.0% or more based on the total weight of the formulation.

In some embodiments, the silicone oil reduces tackiness of the topical formulation. In some instances, the silicone oil is Dimethicone DM350 (medium viscosity). In some instances, the Dimethicone DM350 is present in an amount of at least 3.0% or more based on the total weight of the formulation.

In some embodiments, the natural antioxidant and the lipid preservative is Tocopherol (Vitamin E). In some instances, the Tocopherol is present in an amount of at least 0.5% or more based on the total weight of the formulation.

In some embodiments, the antimicrobial/cosmetic preservative is Euxyl PE 9010, (phenoxyethanol+ethylhexylglycerin). In some instances, the Euxyl PE 9010 is present in an amount of at least 1.0% or more based on the total weight of the formulation.

In some embodiments, any one of the above-described formulations further comprises a fragrance. In some instances, the fragrance is obtained from an essential oil. In some instances, the essential oil is roasted coconut. In some instances, the essential fragrance oil is green tea and cucumber.

In some embodiments, the formulation is a transdermal formulation.

In some embodiments, the formulation is formulated as a broad-spectrum anti-viral agent/adjunct.

In some embodiments, the formulation is formulated as a broad-spectrum anti-cancer agent.

Also described herein are methods of treating Arthritis and Rheumatoid Arthritis; Muscle Ache (Polymyalgia Rheumatica); Peripheral Neuropathies; Diabetic Neuropathies; CNS & Aging Dementias; Multiple Sclerosis; or Palpable cancers, comprising administering to a subject in need thereof any one of the above-described topical formulations.

Also described herein are methods of treating a cancer, comprising administering to a subject in need thereof: a cancer chemotherapy or a cancer gene therapy, and any one of the above-described topical formulations.

Also described herein are methods of treating a disease or disorder, comprising administering to a subject in need thereof: a cell-based therapy, and any one of the above-described topical formulations.

Also provided herein are topical formulations comprising: Andrographolides (ANGL) (i.e., Andrographis paniculata, full spectrum extract); a wetting/hydrating agent; a vegetable oil; a natural triglyceride emollient; a polymeric emulsifier/rheological modifier; a silicone oil; a natural antioxidant; a lipid preservative; and an antimicrobial/cosmetic preservative.

In some embodiments, the ANGL is a powdered extract of Andrographis paniculata (leaf) that is standardized to ≥50% ANGL.

In some embodiments, the ANGL is present in an amount of at least 0.5% % or more based on the total weight of the formulation.

In some embodiments, the ANGL is present in an amount of at least 1.0% or more based on the total weight of the formulation.

In some embodiments, the ANGL is present in an amount of at least 1.5% or more based on the total weight of the formulation.

In some embodiments, the ANGL is present in an amount of at least 2.0% or more based on the total weight of the formulation.

In some embodiments, the wetting/hydrating agent is glycerin, and wherein the wetting/hydrating agent further comprises distilled water. In some instances, the glycerin is present in an amount of at least 6.0% (1:3, ANGL to glycerin) or more based on the total weight of the formulation

In some embodiments, the vegetable oil is Avocado Oil, Macadamia Nut Oil, or Olive Oil. In some instances, the vegetable oil is present in an amount of at least 3.0% or more based on the total weight of the formulation.

In some embodiments, the natural triglyceride emollient is Caprylic/Capric triglyceride. In some instances, the Caprylic/Capric triglyceride is present in an amount of at least 4.0% or more based on the total weight of the formulation.

In some embodiments, the polymeric emulsifier/rheological modifier is Sodium Acrylate/Sodium Acryloyldimethyl Taurate Copolymer-C15-19 Alkane-Polyglyceryl-6 Laurate-Polyglycerin-6. In some instances, the polymeric emulsifier/rheological modifier is present in an amount of at least 3.0% or more based on the total weight of the formulation.

In some embodiments, the silicone oil reduces tackiness of the topical formulation. In some instances, the silicone oil is Dimethicone DM350 (medium viscosity). In some instances, the Dimethicone DM350 is present in an amount of at least 3.0% or more based on the total weight of the formulation.

In some embodiments, the natural antioxidant and the lipid preservative is Tocopherol (Vitamin E). In some instances, the Tocopherol is present in an amount of at least 0.5% or more based on the total weight of the formulation.

In some embodiments, the antimicrobial/cosmetic preservative is Euxyl PE 9010, (phenoxyethanol+ethylhexylglycerin). In some instances, the Euxyl PE 9010 is present in an amount of at least 1.0% or more based on the total weight of the formulation.

In some embodiments, the topical formulation of any one of the preceding embodiments, further comprises a fragrance. In some instances, the fragrance is obtained from an essential oil. In some instances, the essential oil is roasted coconut.

In some embodiments, the topical formulation of any one of the preceding embodiments is formulated as a transdermal formulation.

In some embodiments, the topical formulation of any one of the preceding embodiments is formulated as a broad-spectrum anti-viral agent/adjunct.

In some embodiments, the topical formulation of any one of the preceding embodiments is formulated as a broad-spectrum anti-cancer agent.

Also provide herein are methods of treating Arthritis and Rheumatoid Arthritis; Muscle Ache (Polymyalgia Rheumatica); Peripheral Neuropathies; Diabetic Neuropathies; CNS & Aging Dementias; Multiple Sclerosis; or Palpable cancers, comprising administering to a subject in need thereof the topical formulation of any one of the preceding embodiments.

Also provide herein are methods of treating a cancer, comprising administering to a subject in need thereof: a cancer chemotherapy or a cancer gene therapy, and the topical formulation of any one of the preceding embodiments.

Also provide herein are methods of treating a disease or disorder, comprising administering to a subject in need thereof: a cell-based therapy, and the topical formulation of any one of the preceding embodiments.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A and 1B are images showing an Andrographis extract (50 mg/1.0 ml carrier) solubilization test in two defined cosmetic formulations: FIG. 1A, a light body Lotion; and FIG. 1B, a luxurious Cream prepared with NatureMuls emulsifier according to a Hot Process procedure.

FIGS. 2A and 2B are images showing that Andrographis (˜50 mg) solubilization was visibly poor in (#1) liposome, (#2) phytosome, and (#3) hyaluronic emulsion gel constituents, in comparison with the Gel-Crème (#4). FIG. 2B is an enlargement of FIG. 2A.

FIGS. 3A and 3B are images showing the steps of the “Cold Process” method for Andrographis (ANGL) Solubilization and its Oil-in-Water Emulsification at room remperatures. FIG. 2A shows that the powdered Andrographis API is first wetted with Glycerol at a 3:1 ratio (wt/wt; Glyceral:Andro); followed by solubilization/hydration with dH₂O into an herbal tea/Aqueous Phase (at room temp.). FIG. 2B shows that the Oil Phase components were weighed, combined, and added to the Aqueous Phase, followed by slow mixing of the two phases; followed thereafter by a high-shear emulsification step.

FIG. 4 are images of an elderly male's back showing improvement of overall skin condition after treatment with a formulation comprising Andrographolide.

### DETAILED DESCRIPTION

As noted above, compositions comprising andrographolides are not that bioavailable. Accordingly, provided herein are compositions and pharmaceutical formulations for topical or transdermal delivery (e.g., creams, balms, ointments, lotions) of andrographolides and/or epigallocatechin gallate. Also described herein are methods of making and using these compositions and formulations.

Compositions and Pharmaceutical Formulations Comprising Andrographolides (ANGL) and/or Epigallocatechin Gallate (EGCG)

### Active Ingredient:

### Andrographolide

In some embodiments, the active ingredient of the compositions and formulations described herein is Andrographolide. “Andrographolide” is a diterpene lactone compound extracted from Andrographis paniculata (Burm. f) Nees (A. paniculata), literally ‘king of bitters.’ A. paniculata is an herbaceous plant belonging to the Family Acanthaceae. A. paniculata possesses potent anti-inflammatory activity and contains several compounds, one of which is andrographolide. Andrographolide is obtained either by extracting it from, Andrographis paniculata (aka, Senshinren, Green Chireta, Kalmegh) and then purifying it, or purchasing a commercially available standardized product.

In some embodiments, the compositions and formulations described herein include a high-quality commercial powdered extract of Andrographis paniculata (leaf) standardized to ≥50% Andrographolides (ANGL): specifications greater than about 20%, greater than about 30%, greater than about 40%, or greater than about 50% ANGL, as certified under US/ISO testing standards. The bioactive andrographolides (ANGL) can be andrographolide, deoxyandrographolide, neoandrographolide, or a mixture thereof, contained within an extract of a plant of the genus Andrographis (e.g., Andrographis paniculata).

In some embodiments, the compositions and formulations described herein include 50% ANGL bioactives (i.e., a high quality standardized powered extract) in an amount ranging from about 1% to about 5% wt/wt (that is, 0.5-2.5 grams ANGL diterpenes per 100 ml final product). For example, in some instances the compositions and formulations described herein include 50% ANGL powdered extract in an amount of 1.0%-3.0% wt/wt. In some embodiments, the compositions and formulations described herein include 50% ANGL in an amount of 2.0% wt/wt, yielding ANGL as API at 1.0%.

### Epigallocatechin Gallate

In some embodiments, the active ingredient of the compositions and formulations described herein is Epigallocatechin-3-Gallate. “Epigallocatechin Gallate” is a prized catechin compound extracted from Camellia sinensis (C. sinensis), a species of evergreen shrub or small tree in the flowering plant family Theaceae. Its leaves, leaf buds, and stems can be used to produce tea. Epigallocatechin Gallate extracted from C. sinensis has potent antioxidant, anti-tumor, anti-inflammatory, anti-dementia, and anti-viral activities. Epigallocatechin Gallate is obtained either by extracting it from, for instance, C. sinensis, and then purifying it, or purchasing a standardized commercially available product.

In some embodiments, the compositions and formulations described herein include a high-quality commercial powdered extract of Epigallocatechin Gallate. In these embodiments, the compositions and formulations described herein include highly pure Epigallocatechin Gallate, e.g., powder having a purity of greater than about 80%, greater than about 85%, greater than about 90%, greater than about 91%, greater than about 92%, greater than about 93%, or greater than about 94%). In some instances, the Epigallocatechin Gallate is certified at ≥94% pure, as is obtained from Healthy Origins Teavigo.

In some embodiments, the compositions and formulations described herein include 94% EGCG (i.e., high quality standardized powered extract) in an amount of about 0.5% to about 2.5% wt/wt (e.g., 0.5-2.5 grams EGCG per 100 ml final product). The compositions and formulations described herein include 94% EGCG in an amount of 0.5%-2.5% wt/wt, practical carrying capacity. In some embodiments, the compositions and formulations described herein include 94% EGCG in an amount of 1.0% wt/wt.

### Combinations of EGCG and Alpha-Lipoic Acid (ALA)

In some embodiments of the compositions and formulations described herein featuring EGCG as the primary active ingredient, also include naturally occurring alpha-lipoic acid (ALA). ALA is included in pure form as a co-antioxidant, which is known to protect the EGCG catechin from oxidation. Alpha-lipoic acid is an organosulfur compound derived from caprylic acid (octanoic acid). Alpha-lipoic acid has strong antioxidant/co-antioxidant properties, which may reduce inflammation and skin aging, promote healthy nerve function, lower heart disease risk factors, and slow the progression of memory loss disorders. Alpha-lipoic acid is made naturally in the body and also found in foods (e.g., such as red meat, carrots, beets, spinach, broccoli, and potatoes). In some embodiments including EGCG as the API, the compositions and formulations described herein include ALA (powered extract) as a co-antioxidant in an amount of about 0.1% to about 1% wt/wt (e.g., 0.1 to 1 grams ALA per 100 ml final product). For example, in some instances the compositions and formulations described herein include EGCG at 1% and ALA as a co-antioxidant in an approximately equimolar amount of about 0.6% wt/wt.

### Combinations of ANGL and EGCG

In some embodiments, the compositions and formulations described herein include a synergistic combination of bioactive ingredients. For example, the compositions and formulations may include a combination of both ANGL and EGCG. However, in this particular case, the (EGCG-protective) sulfur-containing ALA is not included, as the Andrographolides would tend to interact covalently/negatively with the sulfur molecules of the ALA. In these instances, the compositions and formulations including both EGCG and ANGL for transdermal co-delivery, may utilize various API ratios that remain within the carrying capacity, which diminishes beyond ˜5% total API.

### Wetting/Hydrating Agent

The compositions and formulations described herein can also include one or more wetting/hydrating agents. A wetting agent (also referred to as a levigating agent) is a liquid used to displace the film of air that exists on the surface of dry powders. Every powder ingredient has a thin layer of air creating a barrier that may hinder the uniform mixing with the base (vehicle) used for a compounded formulation. The wetting agent removes this film of surface air by shear mechanical force and surrounds the powder. A wetting agent therefore facilitates mixing of insoluble powders in liquids and semisolids and may improve drug solubility. This helps facilitate uniform mixing to incorporate the ingredient into the base more evenly, avoid clumping and improve content uniformity of the final preparation. See, Lemus & Rhoads, 2021 PCCA blog.

In some instances, the wetting/hydrating agent is a polyol. In some instances, the polyol is a triol (such as, glycerin/glycerol), a tetraol (such as, pentaerythritol), a pentaol (such as, xylitol). In some instances, 1,3 propanediol (preferred a plant-derived alternative to synthetic propylene glycol) can be used in combination with pure glycerin to reduce tackiness, improve carrying capacity, and/or increase product stability.

In some embodiments, the compositions and formulations described herein include 1:2 to 1:5 (active ingredient:hydrating agent). In some instances about 1:2 (active ingredient:glycerin) is utilized in the compositions and formulations described herein. In some instances about 1:3 (active ingredient:glycerin) is utilized in the compositions and formulations described herein. In some instances about 1:4 (active ingredient:glycerin) is utilized in the compositions and formulations described herein. In some instances about 1:5 (active ingredient:glycerin) is utilized in the compositions and formulations described herein.

In some instances, the compositions and formulations described herein include a hydrating agent in an amount of about 5% to about 10% wt/wt. For example, in some instances the compositions and formulations described herein include glycerin as the wetting/hydrating agent in an optimum amount of about 7% wt/wt.

### Butters/Oils

The compositions and formulations described herein can also include one or more butters and/or oils. In some instances, the butter or oil is a vegetable oil or vegetable butter. In some instances, the butter or oil is an olive oil or olive butter. In some instances, the butter or oil is an avocado oil or avocado butter. In some instances, the butter or oil is a macadamia nut oil or macadamia nut butter.

In some embodiments, the compositions and formulations described herein include butter or oil (e.g., Avocado Oil, Macadamia Nut Oil, or Olive Oil) in an amount of about 1% to about 5% wt/wt. For example, in some instances the compositions and formulations described herein include refined avocado oil in an amount of 3.0% wt/wt.

### Natural Triglyceride Emollient

The compositions and formulations described herein can also include one or more natural triglyceride emollients for protecting, moisturizing, and lubricating the skin. In some embodiments, the natural triglyceride emollients are caprylic/capric triglycerides, which are made from naturally occurring fatty acids and can be derived from coconut oil. Other examples of natural triglyceride emollients include a variety of glyceryl esters.

In these embodiments, the compositions and formulations described herein include a natural triglyceride emollient and solubilizing compound in amount of about 1% to about 7% wt/wt. In some embodiments, the compositions and formulations described herein include caprylic/capric triglycerides derived from purified coconut oil—purified caprylic, C8, and capric, C10, fatty acids chemically re-esterified with glycerin—in an amount of 4.0% wt/wt.

### Polymeric Emulsifier & Rheological Modifier

The compositions and formulations described herein can also include one or more polymeric emulsifier & rheological modifiers. Polymeric emulsifier and rheological modifiers are used to emulsify oils in water and to help the overall viscosity of the formulation. In some embodiments, the polymeric emulsifier & rheological modifier is plant-based and renewable, China compliant (IECIC list), Halal certified, and vegan-based. In some embodiments, the polymeric emulsifier & rheological modifier that is used is ideal for cold emulsifying processes (i.e., emulsifying at room temperature). In some embodiments, the polymeric emulsifier & rheological modifier that is Sodium Acrylate/Sodium Acryloyldimethyl Taurate Copolymer-C15-19 Alkane-Polyglyceryl-6 Laurate-Polyglycerin-6 (sold as “SEPILIFET™” from Seppic).

In some embodiments, the compositions and formulations described herein include the polymeric emulsifier & rheological modifier in an amount of about 1% to about 5% wt/wt. For example, in some instances the compositions and formulations described herein include Sodium Acrylate/Sodium Acryloyldimethyl Taurate Copolymer-C15-19 Alkane-Polyglyceryl-6 Laurate-Polyglycerin-6 in an amount of 3.0% wt/wt.

### Silicone Oil

The compositions and formulations described herein can also include one or more medium viscosity silicone oils for reducing the tackiness of the formulation. These silicone oils are also helping for forming a barrier over the skin or hair, reducing moisture loss, and smoothing over bumps and pores. Examples of medium viscosity silicone oils include polydimethylsiloxane, also known as dimethylpolysiloxane or dimethicone (e.g., Dimethicone DM350 CS).

In some embodiments, the compositions and formulations described herein include the medium viscosity silicone oil in an amount of about 1% to about 5% wt/wt. For example, in some instances the compositions and formulations described herein include Dimethicone DM350 CS in an amount of 3.0% wt/wt.

### Natural Antioxidants

The compositions and formulations described herein can also include one or more antioxidants. In some embodiments, the antioxidant is also a lipid preservative. In some embodiments, the antioxidant is Tocopherol (Vitamin E). Other examples of antioxidants include, stilbenoid (Resveratrol), Niacinamide (vitamin B3), Carotenoids, and Rosemary oleoresin.

In some embodiments, the compositions and formulations described herein include the natural antioxidant in an amount of about 0.1% to about 1% wt/wt. For example, in some instances the compositions and formulations described herein include Tocopherol (Vitamin E) in an amount of 0.5% wt/wt.

### Antimicrobial and Cosmetic Preservative

The compositions and formulations described herein can also include one or more antimicrobial and cosmetic preservatives. In some embodiments, the antimicrobial and cosmetic preservative is Euxyl PE 9010, (phenoxyethanol+ethylhexylglycerin). Other examples of antimicrobial and cosmetic preservative include, Liquid Germall.

In some embodiments, the compositions and formulations described herein include the antimicrobial and cosmetic preservative in an amount of about 0.5% to about 2% wt/wt. For example, in some instances the compositions and formulations described herein include Euxyl PE 9010 in an amount of about 1.0% wt/wt.

### Fragrances

In some embodiments, one or more fragrances are also optionally included in the compositions and formulations described herein. Examples of fragrances include any essential oil that imparts a pleasant smell. In some embodiments, the fragrance is Roasted Coconut. Other examples include, Lavender, Lemon, Eucalyptus, any citrus fragrances (like, Orange, Grapefruit), Ginger, Tea Tree, Mint, Rosemary, Cedarwood, Ylang Ylang, or any combination. Any of the essential oils are included in the compositions/formulation as a CO₂ extract.

In some embodiments, the compositions and formulations described herein include the fragrance in the form of a CO₂ extract essential oil in an amount of about 0.1% to about 1% wt/wt. For example, in some instances the compositions and formulations described herein include a CO₂ extract of Roasted Coconut Oil in an amount of 0.5% wt/wt. In another example, the compositions and formulations described herein include a CO₂ extract of Green Tea and Cucumber Fragrance Oil in an amount of 0.2% wt/wt.

### Glutathione

In some embodiments, the compositions and formulations described herein do not include confounding cosmeceutical ingredients, such as glutathione.

In some embodiments, the compositions and formulations as described herein include a gel-crème as a vehicle carrier for one or more active ingredient. The gel-crème of the compositions and formulations comprising the one or more active ingredients comprises the following: d-H₂O at a concentration of about 700 mg/ml to about 800 mg/ml (e.g., about 700 mg/ml, about 710 mg/ml, about 720 mg/ml, about 730 mg/ml, about 740 mg/ml, about 750 mg/ml, about 760 mg/ml, about 770 mg/ml, about 780 mg/ml, about 790 mg/ml, about 800 mg/ml, or any increment in between); glycerin at concentration of about 65 mg/ml to about 75 mg/ml (e.g., about 65 mg/ml, about 66 mg/ml, about 67 mg/ml, about 68 mg/ml, about 69 mg/ml, about 70 mg/ml, about 71 mg/ml, about 72 mg/ml, about 73 mg/ml, about 74 mg/ml, about 75 mg/ml, or any increment in between); caprylic/capric triglycerides derived from purified coconut oil at a concentration of about 35 mg/ml to about 45 mg/ml (e.g., about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, or any increment in between); avocado oil at a concentration of about 25 mg/ml to about 35 mg/ml (e.g., about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, or any increment in between); dimethicone 350 at a concentration of about 25 mg/ml to about 35 mg/ml (e.g., about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, or any increment in between); Sodium Acrylate/Sodium Acryloyldimethyl Taurate Copolymer-C15-19 Alkane-Polyglyceryl-6 Laurate-Polyglycerin-6 is at a concentration of about 25 mg/ml to about 35 mg/ml (e.g., about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, or any increment in between); vitamin E (i.e., tocopherol) at a concentration of about 1 mg/ml to about 10 mg/ml (e.g., about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, or any increment in between); Euxyl PE 9010 at a concentration of about 5 mg/ml to about 15 mg/ml (e.g., about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, or any increment in between); and a fragrance at a concentration of about 1 mg/ml to about 10 mg/ml (e.g., about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, or any increment in between).

In some embodiments, the active ingredient of the compositions and formulations is ANGL and the concentration of ANGL is about 5 mg/ml to about 30 mg/ml (e.g., about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, or any increment in between).

In some embodiments, the active ingredient of the compositions and formulations is a combination of EGCG and ALA, wherein the concentration of EGCG is about 5 mg/ml to about 20 mg/ml (e.g., about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, or any increment in between) and the concentration of ALA is about 1 mg/ml to about 15 mg/ml (e.g., about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, or any increment in between).

In some embodiments, the combination of active ingredients may include both ANGL and EGCG in varying amounts-sans the ALA co-antioxidant—while remaining within the determined “carrying capacity” (total API<5% of the transdermal gel-crème), beyond which the transdermal delivery and vanishing crème properties are diminished.

Any of the therapeutic compositions disclosed herein can be formulated for sale in the US, imported into the US, and/or exported from the US. The pharmaceutical compositions can be included in a kit, a container, a pack, a dispenser, and/or with instructions for administration.

### Methods of Making

Also provided herein are methods for making the compositions and formulations described herein.

In some embodiments, the compositions and formulations described herein are made using cold emulsion processes, which are well known in the art.

In general, a cold emulsion process eliminates the need for using heat during the emulsification process. In this method, the ingredients are combined at or near room temperature, hence the term “cold process.” Traditionally, lotions/creams, are made using heat to melt the emulsifier and combine the water and oil phases. However, in cold processes, emulsifiers are chosen specifically for their ability to emulsify at lower temperatures, allowing for the creation of lotions/creams without the need for heat.

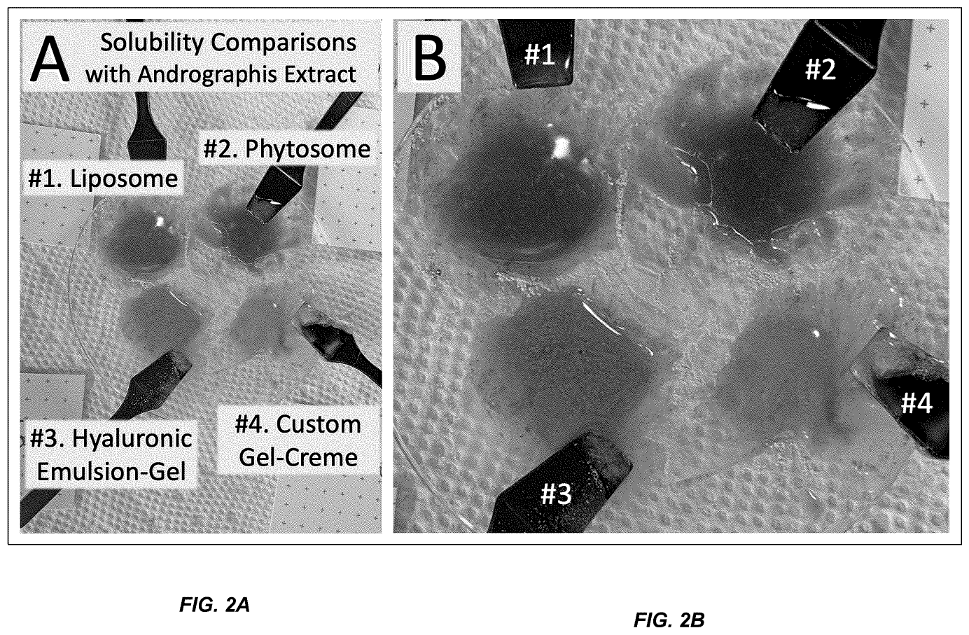

As described herein, the compositions and formulations described herein are made using an emulsification process that is performed at room temperature—i.e., Cold Process (see FIG. 2). The initial wetting of the standardized herbal powder (e.g., ANGL, ECGC, ALA, or a specified combination thereof) with Glycerin is followed by further solubilization in d-H₂O to prepare the Aqueous Phase (FIG. 2A). Mixing of the Aqueous Phase solution with the polymeric Oil Phase components, is then followed by high-shear (Oil-in-Water) emulsification to form the stable active ingredient (e.g., ANGL, ECGC, ALA, or a specified combination thereof)-loaded Gel-Crème product (FIG. 2B). In these embodiments, the methods utilized herein do not require heating or pre-heating of product components. The active ingredients, such as bioactive andrographolide diterpenes (ANGL) and ALA, are degraded significantly by high temperatures, which are commonly utilized in so-called Hot Process formulas and emulsification procedures.

### Methods of Using the Compositions and Formulations Described Herein

As noted above, despite having therapeutic utility, both Andrographolides and EGCG exhibit poor oral bioavailability (absolute bioavailability of ANGL is only about 2.67% and EGCG is only about 0.16%). ANGL diterpenes also undergo extensive biotransformation through phase I hydroxylation and phase II conjugation during hepatobiliary first-pass metabolism, before reaching the systemic circulation.

Accordingly, in some embodiments, the compositions and formulations described herein are for topical/transdermal use. In contrast to the limitations of oral administration, the topical/transdermal delivery of compositions or formulations comprising ANGL, ECGC, ALA, or a specified combination thereof described herein enhance delivery of the active ingredient to a patient at three distinct levels. Specifically, the compositions and formulations permit: (1) highly localized topical drug delivery; (2) deep-tissue (muscle and/or joint) delivery; and (3) systemic delivery. These three levels of enhanced API delivery correspond in place and time with reported patient responses: that is, immediate relief, delayed deep-relief, and systemic inflammation/pain relief (enhanced well-being), respectively.

In some embodiments, the compositions or formulations comprising ANGL, ECGC, ALA, or a specified combination thereof described herein can be utilized in methods of treating various diseases, disorders, or conditions, wherein the methods include administering to a subject in need thereof a therapeutic amount of the compositions or formulations comprising ANGL, ECGC, ALA, or a specified combination thereof described herein for a time sufficient to result in a therapeutic result.

In some embodiments, the methods include treating Arthritis and Rheumatoid Arthritis; Muscle Ache (Polymyalgia Rheumatica); Peripheral Neuropathies; Intervertebral disc disease (IVDD); Diabetic Neuropathies; CNS Aging Dementias; Multiple Sclerosis; or Palpable cancers (e.g., any cancer that can be felt by touch, usually present in lymph nodes, skin, or other organs of the body such as the liver or colon), comprising administering to a subject in need thereof the compositions and formulations described herein. In some embodiments the palpable cancer is a skin cancer (e.g., basal cell carcinoma, squamous cell carcinoma, or melanoma).

Also provided herein are methods of improving cancer therapies (e.g., a cancer chemotherapy or a cancer gene therapy) by additionally administering to a subject in need thereof the compositions and formulations described herein. For example, in some embodiments, a subject who is being treated for a palpable cancer (e.g., a skin cancer) with a cancer chemotherapy or a cancer gene therapy can additionally be administered the compositions and formulations described herein. In some embodiments, the compositions and formulations described herein are formulated as a broad-spectrum anti-cancer agent.

Also provided herein are methods of improving cell-based therapies by additionally administering to a subject in need thereof the compositions and formulations described herein.

Also included in here are methods of treating a viral infection. In some embodiments, the compositions and formulations described herein are formulated as broad-spectrum anti-viral agents/adjuncts.

The terms “administer”, “administering”, or “administration” as used herein refers to administering the compositions and formulations described herein to a subject using any art-known method, e.g., topically administering the composition/formulation by applying the composition/formulation to the surface of a subject's skin.

The terms “treat” or “treating” refer to accomplishing one or more of the following: (a) reducing the severity of a disease, disorder, condition, or symptom; (b) limiting the development of symptoms characteristic of the disease, disorder, or condition being treated; (c) limiting worsening of symptoms characteristic of the disease, disorder, or condition being treated; (d) limiting recurrence of the disease, disorder, or condition in subjects that have previously had the disease, disorder, or condition; and (e) limiting recurrence of symptoms in subjects that were previously symptomatic for the disease, disorder, or condition. Any of the present methods can include a step of identifying a patient in need of treatment. For example, a subject can be examined and/or subjected to clinical tests in order to determine whether they have, for example, arthritis and rheumatoid arthritis; muscle ache (Polymyalgia Rheumatica); a peripheral neuropathy; a diabetic neuropathy; a CNS & aging dementia; multiple sclerosis; or a palpable cancer.

The methods herein include administration of an effective amount of the composition/formulation to achieve the desired or stated effect. Specific dosage and treatment regimens for any particular subject will depend upon a variety of factors, including condition to be addressed, the age, body weight, general health status, sex, diet, time of administration, rate of excretion, combination with any other therapeutic agent, the severity and course of the disease, condition or symptoms, the subject's disposition to the disease, disorder, condition, or symptoms, and the judgment of the treating physician.

The methods described herein include topically administering to the subject a therapeutically effective amount of the composition/formulation described herein (see, e.g., Messire, Gatien et al. “Antioxidant Effects of Catechins (EGCG), Andrographolide, and Curcuminoids Compounds for Skin Protection, Cosmetics, and Dermatological Uses: An Update.” Antioxidants (Basel, Switzerland) vol. 12,7 1317. 21 Jun. 2023). In some embodiments, the methods described herein include administering to a subject a dose of about 5 mg to about 1500 mg of ANGL, including any increment in between. In some embodiments, the methods described herein include administering to a subject a dose of about 5 mg to about 1250 mg of ANGL, including any increment in between. In some embodiments, the methods described herein include administering to a subject a dose of about 5 mg to about 1000 mg of ANGL, including any increment in between. In some embodiments, the methods described herein include administering to a subject a dose of about 5 mg to about 750 mg of ANGL, including any increment in between. In some embodiments, the methods described herein include administering to a subject a dose of about 5 mg to about 500 mg of ANGL, including any increment in between. In some embodiments, the methods described herein include administering to a subject a dose of about 5 mg to about 250 mg of ANGL, including any increment in between. In some embodiments, the methods described herein include administering to a subject a dose of about 10 mg to about 250 mg of ANGL. In some embodiments, the each dose that is administered to a subject includes about 5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg of ANGL, about 300 mg of ANGL, about 350 mg of ANGL, about 400 mg of ANGL, about 450 mg of ANGL, about 500 mg of ANGL, about 550 mg of ANGL, about 600 mg of ANGL, about 650 mg of ANGL, about 700 mg of ANGL, about 750 mg of ANGL, about 800 mg of ANGL, about 850 mg of ANGL, about 900 mg of ANGL, about 950 mg of ANGL, about 1000 mg of ANGL, about 1250 mg of ANGL, about 1500 mg of ANGL, or any increment in between.

In some embodiments, the methods include administering to a patient about 5 ml to about 50 ml of the formulation/composition described herein (e.g., about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, about 50 ml, or any increment in between), wherein the formulation/composition has a concentration of about 1 mg/ml to about 50 mg/ml (e.g., about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, or any increment in between) of ANGL.

In some embodiments, the composition/formulation is administered to a subject ad libitum. In some instances, the total amount of ANGL administered to a subject does not exceed about 1500 mg per day. In some instances, the total amount of ANGL administered to a subject does not exceed about 1000 mg per day. In some instances, the total amount of ANGL administered to a subject does not exceed about 750 mg per day. In some instances, the total amount of ANGL administered to a subject does not exceed about 500 mg per day. In some instances, the total amount of ANGL administered to a subject does not exceed about 400 mg per day. In some instances, the total amount of ANGL administered to a subject does not exceed about 390 mg per day. In some instances, the total amount of ANGL administered to a subject does not exceed about 380 mg per day. In some instances, the total amount of ANGL administered to a subject does not exceed about 370 mg per day. In some instances, the total amount of ANGL administered to a subject does not exceed about 360 mg per day. In some instances, the total amount of ANGL administered to a subject does not exceed about 350 mg per day. In some instances, the total amount of ANGL administered to a subject does not exceed about 340 mg per day. In some instances, the total amount of ANGL administered to a subject does not exceed about 330 mg per day. In some instances, the total amount of ANGL administered to a subject does not exceed about 320 mg per day. In some instances, the total amount of ANGL administered to a subject does not exceed about 310 mg per day. In some instances, the total amount of ANGL administered to a subject does not exceed about 300 mg per day. In some instances, the total amount of ANGL administered to a subject does not exceed about 290 mg per day. In some instances, the total amount of ANGL administered to a subject does not exceed about 280 mg per day. In some instances, the total amount of ANGL administered to a subject does not exceed about 270 mg per day. In some instances, the total amount of ANGL administered to a subject does not exceed about 260 mg per day. In some instances, the total amount of ANGL administered to a subject does not exceed about 250 mg per day. In some instances, the total amount of ANGL administered to a subject does not exceed about 240 mg per day. In some instances, the total amount of ANGL administered to a subject does not exceed about 230 mg per day. In some instances, the total amount of ANGL administered to a subject does not exceed about 220 mg per day. In some instances, the total amount of ANGL administered to a subject does not exceed about 210 mg per day. In some instances, the total amount of ANGL administered to a subject does not exceed about 200 mg per day. In some instances, the total amount of ANGL administered to a subject does not exceed about 190 mg per day. In some instances, the total amount of ANGL administered to a subject does not exceed about 180 mg per day. In some instances, the total amount of ANGL administered to a subject does not exceed about 170 mg per day. In some instances, the total amount of ANGL administered to a subject does not exceed about 160 mg per day. In some instances, the total amount of ANGL administered to a subject does not exceed about 150 mg per day. In some instances, the total amount of ANGL administered to a subject does not exceed about 140 mg per day. In some instances, the total amount of ANGL administered to a subject does not exceed about 130 mg per day. In some instances, the total amount of ANGL administered to a subject does not exceed about 120 mg per day. In some instances, the total amount of ANGL administered to a subject does not exceed about 110 mg per day. In some instances, the total amount of ANGL administered to a subject does not exceed about 100 mg per day.

Also provided herein are methods of treating spinal arthritis & seborrheic keratosis in a subject in need thereof, wherein 5-7 ml ANGL Gel-Crème (at a concentration of 10 mg/ml) is applied to the subject's back & along the spine at least once per day.

Also provided herein are methods of treating arthritic knees in a subject in need thereof, wherein 4-5 ml ANGL Gel-Crème (at a concentration of 10 mg/ml) is applied to the subject's knees at least twice per day.

Also provided herein are methods of treating neuropathies of the toes in a subject in need thereof, wherein 1-2 ml ANGL Gel-Crème (at a concentration of 10 mg/ml) is applied to the subject's toes at least twice per day.

The methods described herein include topically administering to the subject a therapeutically effective amount of the composition/formulation described herein (see, e.g., Andreu-Fernindez, Vicente et al. “Bioavailability of Epigallocatechin Gallate Administered With Different Nutritional Strategies in Healthy Volunteers.” Antioxidants (Basel, Switzerland) vol. 9,5 440. 19 May. 2020). In some embodiments, the methods described herein include administering to a subject a dose of about 5 mg to about 1500 mg of EGCG, including any increment in between. In some embodiments, the methods described herein include administering to a subject a dose of about 5 mg to about 1250 mg of EGCG, including any increment in between. In some embodiments, the methods described herein include administering to a subject a dose of about 5 mg to about 1000 mg of EGCG, including any increment in between. In some embodiments, the methods described herein include administering to a subject a dose of about 5 mg to about 750 mg of EGCG, including any increment in between. In some embodiments, the methods described herein include administering to a subject a dose of about 5 mg to about 500 mg of EGCG, including any increment in between. In some embodiments, the methods described herein include administering to a subject a dose of about 5 mg to about 250 mg of EGCG, including any increment in between. In some embodiments, the methods described herein include administering to a subject a dose of about 10 mg to about 250 mg of EGCG. In some embodiments, the each dose that is administered to a subject includes about 5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg of EGCG, about 300 mg of EGCG, about 350 mg of EGCG, about 400 mg of EGCG, about 450 mg of EGCG, about 500 mg of EGCG, about 550 mg of EGCG, about 600 mg of EGCG, about 650 mg of EGCG, about 700 mg of EGCG, about 750 mg of EGCG, about 800 mg of EGCG, about 850 mg of EGCG, about 900 mg of EGCG, about 950 mg of EGCG, about 1000 mg of EGCG, about 1250 mg of EGCG, about 1500 mg of EGCG, or any increment in between.

In some embodiments, the methods include administering to a patient about 3 ml to about 50 ml of the formulation/composition described herein (e.g., about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, about 50 ml, or any increment in between), wherein the formulation/composition has a concentration of about 1 mg/ml to about 50 mg/ml (e.g., about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, or any increment in between) of EGCG.

In some embodiments, the composition/formulation is administered to a subject ad libitum. In some instances, the total amount of EGCG administered to a subject does not exceed about 1500 mg per day. In some instances, the total amount of EGCG administered to a subject does not exceed about 1000 mg per day. In some instances, the total amount of EGCG administered to a subject does not exceed about 750 mg per day. In some instances, the total amount of EGCG administered to a subject does not exceed about 500 mg per day. In some instances, the total amount of EGCG administered to a subject does not exceed about 400 mg per day. In some instances, the total amount of EGCG administered to a subject does not exceed about 390 mg per day. In some instances, the total amount of EGCG administered to a subject does not exceed about 380 mg per day. In some instances, the total amount of EGCG administered to a subject does not exceed about 370 mg per day. In some instances, the total amount of EGCG administered to a subject does not exceed about 360 mg per day. In some instances, the total amount of EGCG administered to a subject does not exceed about 350 mg per day. In some instances, the total amount of EGCG administered to a subject does not exceed about 340 mg per day. In some instances, the total amount of EGCG administered to a subject does not exceed about 330 mg per day. In some instances, the total amount of EGCG administered to a subject does not exceed about 320 mg per day. In some instances, the total amount of EGCG administered to a subject does not exceed about 310 mg per day. In some instances, the total amount of EGCG administered to a subject does not exceed about 300 mg per day. In some instances, the total amount of EGCG administered to a subject does not exceed about 290 mg per day. In some instances, the total amount of EGCG administered to a subject does not exceed about 280 mg per day. In some instances, the total amount of EGCG administered to a subject does not exceed about 270 mg per day. In some instances, the total amount of EGCG administered to a subject does not exceed about 260 mg per day. In some instances, the total amount of EGCG administered to a subject does not exceed about 250 mg per day. In some instances, the total amount of EGCG administered to a subject does not exceed about 240 mg per day. In some instances, the total amount of EGCG administered to a subject does not exceed about 230 mg per day. In some instances, the total amount of EGCG administered to a subject does not exceed about 220 mg per day. In some instances, the total amount of EGCG administered to a subject does not exceed about 210 mg per day. In some instances, the total amount of EGCG administered to a subject does not exceed about 200 mg per day. In some instances, the total amount of EGCG administered to a subject does not exceed about 190 mg per day. In some instances, the total amount of EGCG administered to a subject does not exceed about 180 mg per day. In some instances, the total amount of EGCG administered to a subject does not exceed about 170 mg per day. In some instances, the total amount of EGCG administered to a subject does not exceed about 160 mg per day. In some instances, the total amount of EGCG administered to a subject does not exceed about 150 mg per day. In some instances, the total amount of EGCG administered to a subject does not exceed about 140 mg per day. In some instances, the total amount of EGCG administered to a subject does not exceed about 130 mg per day. In some instances, the total amount of EGCG administered to a subject does not exceed about 120 mg per day. In some instances, the total amount of EGCG administered to a subject does not exceed about 110 mg per day. In some instances, the total amount of EGCG administered to a subject does not exceed about 100 mg per day.

Treatment methods can include a single administration, multiple administrations, and repeating administration as required for the prophylaxis or treatment of any of the disorders as noted above. The duration of prophylaxis treatment can be a single dosage or the treatment may continue (e.g., multiple dosages), e.g., for weeks, months, years or indefinitely for the lifespan of the subject.

### EXAMPLES

Additional embodiments are disclosed in further detail in the following examples, which are provided by way of illustration and are not in any way intended to limit the scope of this disclosure or the claims.

### Example 1: Creation of a Gel-Crème Formulation with Andrographolide

A light body Lotion; and a luxurious Cream were both prepared using NatureMuls as the emulsifier, according to the following procedures.

A. Hot Process Lotion with NatureMuls

### Phase A.

| | |
|---|---|
| Water | To 100 ml |
| Vegetable Glycerin | 8.0 ml |
| Xanthan Gum (soft) | 0.5 ml |

### Phase B.

| | |
|---|---|
| NatureMuls | 5.0 ml |
| (Candelilla/Jojoba/Rice Bran Polyglyceryl-3 Esters, | |
| Glyceryl Stearate, Cetearyl Alcohol, | |
| Sodium Stearoyl Lactylate) | |
| Murumuru Butter | 2.0 ml |
| Baobab Oil | 3.0 ml |
| Caprylic/Capric Triglyceride | 3.0 ml |
| Isoamyl Cocoate | 2.0 ml |
| Dimethicone 100 cs. | 2.0 ml |

### Phase C

| | |
|---|---|
| Hydrolyzed Baobab Protein | 2.0 ml |
| Tocopherol mixed | 0.5 ml |
| Fragrance Oil | 0.3 ml |
| Euxyl PE 9010 | 1.0 ml |
| (Phenoxyethanol (and) Ethylhexylglycerin) | |

### Phase D.

| | |
|---|---|
| pH Adjuster | q.s |
| (Sodium Hydroxide 30% solution or Citric Acid 50% solution) | |

### Process:

Xanthan gum was combined with glycerin in a slurry and was then added to water. This Phase A mixture was then heated to 80° C.

Components of Phase B and their respective amounts were combined and heated to 80° C.

Phase B was added to Phase A under high shear (high speed (12 k RPM)) until a glossy smooth emulsion formed. This was cooled to under 40° C. Phase C components were combined and added to the Phase A/Phase B mixture under low shear until homogenous.

The pH was then checked and adjusted with Phase D to a pH of approximately 5.0-5.5.

B. Hot Process Body Cream with NatureMuls

### Phase A.

| | |
|---|---|
| Water | To 100 ml |
| Vegetable Glycerin | 8.0 ml |
| Xanthan Gum (soft) | 0.5 ml |

### Phase B.

| | |
|---|---|
| NatureMuls | 5.0 ml |
| (Candelilla/Jojoba/Rice Bran Polyglyceryl-3 Esters, | |
| Glyceryl Stearate, Cetearyl Alcohol, | |
| Sodium Stearoyl Lactylate) | |
| Cetyl Alcohol | 4.0 ml |
| PEG-8 Beeswax | 1.0 ml |
| Murumuru Butter | 3.0 ml |
| Baobab Oil | 3.0 ml |
| Caprylic/Capric Triglyceride | 4.0 ml |
| Isoamyl Cocoate | 3.0 ml |
| Dimethicone 100 cs. | 2.0 ml |

### Phase C.

| | |
|---|---|
| Hydrolyzed Baobab Protein | 2.0 ml |
| Tocopherol mixed | 0.5 ml |
| Essential oil blend | 0.5 ml |
| (French Lavender, Geranium, Roman Chamomile) | |
| Euxyl PE 9010 | 1.0 ml |
| (Phenoxyethanol, Ethylhexylglycerin) | |

### Phase D

| | |
|---|---|
| pH Adjuster | q.s |
| (Sodium Hydroxide 30% solution or Citric Acid 50% solution) | |

### Process:

Xanthan gum was combined with glycerin in a slurry and was then added to water. This Phase A mixture was then heated to 80° C.

Components of Phase B and their respective amounts were combined and heated to 80° C.

Phase B was added to Phase A under high shear (high speed (12 k RPM)) until a glossy smooth emulsion formed. This was cooled to under 40° C. Phase C components were combined and added to the Phase A/Phase B mixture under low shear until homogenous.

The pH was then checked and adjusted with Phase D to a pH of approximately 5.0-5.5.

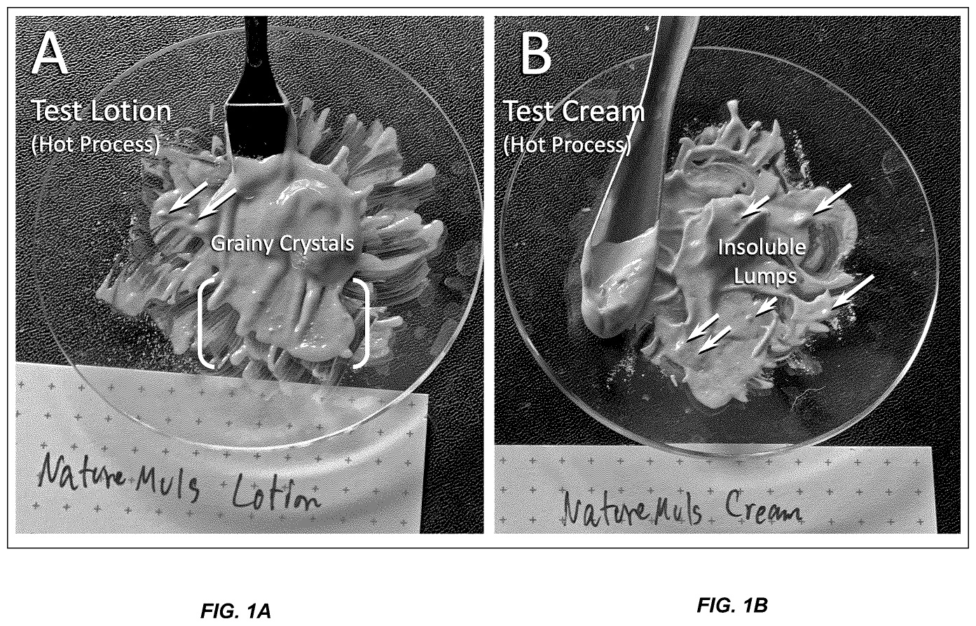

As shown in FIG. 1 A, B, solubilization was relatively poor in both the Test Lotion (A) and the Test Cream (B), in terms of visible crystals (brackets), and clumps (white arrows), as well as resulting texture, graininess, and glide. Additionally, Hot Process emulsifying methods were determined to be less suitable for development, in general, in terms of preserving the medicinal Andro/ANGL bioactivities, as well as Andro/ANGL emulsifying/carrying capacity.

To identify other/better methods and procedures for solubilizing Andrographis herbal powder, several additional approaches were investigated: including, #1, an advanced Liposomal formulation of micronized palmitoethanolamide provided by Rosmolo; #2, a Phytosome (lecithin mixed phospholipid supplement) comprised of phosphatidylcholine, phosphatidylethanolamine, phosphatidylinositol, and glycolipids, provided by Core Med Science (Costa Mesa, CA); #3, a Hyaluronic Emulsion-Gel; and #4, a Custom Gel-Crème. Details of each formulation and the way in which they were made are provided below.

### #1 Liposomal Formulation

The liposomal-containing formulation was prepared by mixing Andrographis herbal powder with a commercially obtained liposomal formulation (e.g., the one sold by Rosmolo, “Liposomal Palmitoylethanolamide 1000 mg+Luteolin 100 mg, Micronized Pea 99% Highly Purified”). First, the liposomal PEA was extracted from the gel caps purchased from Rosmolo. Second, the contents were mixed with Moringa Oil and Black Currant Oil (1:1) to generate a stock solution. Third, the Andrographis herbal powder was mixed with the stock solution using a hot pot procedure.

### #2 Phytosome Formulation

The phytosome-containing formulation was prepared by mixing Andrographis herbal powder with a commercially obtained phytosomal lecithin phospholipid formulation (e.g., the one sold by Core Med Science, “Phosphatidylcholine Supplement, Liposomal PC Complex”). First, the Phytosomal PC Complex was used to help solubilize a dry powder extract of Bacopa monnieri in the context of a ˜50% ethanolic/H2O extract of Hawthorn (Crataegus; this tincture approximating clinical-grade WS1442), creating a bioactive Stock Solution. Subsequently, the Andrographis herbal powder was mixed with the stock solution using a hot pot procedure.

### #3—Hyaluronic Emulsion-Gel (Aloe Vera Emul-Gel (Cold Process))

The Hyaluronic Emulsion-Gel formulation was prepared using a cold process method as outlined below.

### Phase A.

| | |
|---|---|
| Water | To 100 ml |
| Vegetable Glycerin | 5.0 ml |
| Panthenol | 2.0 ml |
| Allantoin | 1.0 ml |
| Betaine (Trimethylglycine) | 2.0 ml |
| Aloe Vera powder (200x) | 0.5 ml |

### Phase B.

| | |
|---|---|
| Lavender Hydrosol | 30.0 ml |
| Cucumber Hydrosol | 15.0 ml |
| Gotu Kola Ext.(liquid) | 5.0 ml |
| Chamomile Ext. (liquid) | 2.0 ml |
| Liquid Germall Plus | 0.5 ml |
| (Propylene Glycol (and) Diazolidinyl Urea (and) | |
| Iodopropynyl Butylcarbamate) | |

### Phase C.

| | |
|---|---|
| Tocopherol mixed | 0.2 ml |
| French Lavender Essential Oil | 4.0 ml |
| Polysorbate-20 | 12.0 ml |

### Phase D.

| | |
|---|---|
| PH. Adjuster to 5.0-5.5 | q.s |

### Phase E.

| | |
|---|---|
| Sodium Hyaluronate (HMW) (High Molecular Weight) | 1.6 ml |

### Process:

Phase A components were combined and heated slightly (around 45° C.-55° C.) until everything was dissolved.

Phase B components were combined and then added to Phase A under low shear (slow speed (4-5 k RPM)).

Phase C components were combined until homogenous; this was then added to Phase A/B under low shear (slow speed (4-5 k RPM)).

The pH was checked and adjusted with Phase D to a final pH of 5.0-5.5.

After pH was adjusted, Phase E was added to Phase A/B/C/D and was allowed to swell overnight or hours.

The mixture was stirred under low shear to break up lumps until a smooth homogenous gel formed.

#4—Custom Gel-Crème (Original Gel-Crème with Sepilife Nude (Cold Process))

### Phase A.

| | |
|---|---|
| Water | To 100 ml |
| Vegetable Glycerin | 6.0 ml |
| Liquid Germall Plus | 0.5 ml |

### Phase B.

| | |
|---|---|
| Caprylic/Capric Triglyceride | 4.0 ml |
| Avocado oil | 3.0 ml |
| Dimethicone 350 cs. | 3.0 ml |
| Sepilife Nude | 3.0 ml |
| (Sodium Acrylate/Sodium Acryloyldimethyl | |
| Taurate Copolymer (and) C15-19 Alkane (and) | |
| Polyglyceryl-6 Laurate (and) Polyglycerin-6) | |
| Roasted Coconut Essential Oil | 0.5 ml |
| Tocopherol mixed | 0.2 ml |

### Process:

Phase A ingredients were combined by mixing under low shear (slow speed (4-5 k RPM)).

Phase B components were combined and then added to Phase A under medium/high shear (medium to high speed (6-12 k RPM)) (until a glossy smooth emulsion formed, and thickened uniformly.

A side-by-side comparison of these prospective carriers is shown in FIG. 2A, B. Both the Liposome and the Phytosome carrier formulations solubilized the powdered Andrographis extract to some extent; however, visible light and dark-clods and particulates were still evident in both the Liposome and the Phytosome/Andrographis admixtures. The Hyaluronic Emulsion-Gel fared poorly in comparison, with numerous insoluble particulates remaining visible. By contrast, the custom Gel-Crème formulation was noticably smoother, incorporating Andrographis rapidly, with a resulting fine grain and a uniform color, and with little insoluble traces remaining after simple mixing. The comparison showed that the Custom Gel-Crème (prepared at Room Temp) was surprisingly & remarkably effective in immediately solubilizing the powdered Andrographis extract, while remaining smooth and creamy to the touch. Thus, the custom/original light Gel-Crème was selected for further development as a transdermal ANGL delivery vehicle.

The Optimized Formulation and Method of Andrographis Incorporation: A method wherein an Andrographis standardized extract is solubilized, emulsified, and incorporated at room temperatures (without heating or pre-heating components) into the composition of a light, minimalistic, fast-penetrating, and bioactive Andrographis Gel-Crème is described.

The Andrographis Gel-Crème formulation was created according to the following Cold Process Method (see components and amounts listed in Table 1 below).

TABLE 1

| | | |
|---|---|---|
| Reagents/Phase | % Formula | 400 g (total wt) |

A: Aqueous Phase

| | | |
|---|---|---|
| Pure Distilled Water | 76.0% | 304 g |
| Pure Glycerin | 7.0% | 28 g |
| Andro/ANGL 50% | 2.0% | 8 g |
| Total (Aqueous Phase) | 85.0% | 340 g |
| B: Oil Phase | | |
| Caprylic/C. Triglycerides | 4.0% | 16 g |
| Refined Avocado Oil | 3.0% | 12 g |
| Dimethicone 350 | 3.0% | 12 g |
| SepilifeNude, Copolymer | 3.0% | 12 g |
| (Emulsifier/Rheol. Blend) | | |
| Roasted Coconut Oil | 0.5% | 2 g |
| Tocopherol (Vitamin E) | 0.5% | 2 g |
| Euxyl PE 9010 | 1.0% | 4 g |
| Total (Oil Phase) | 15% | 60 g |
| Total: | 100% | 400 g |

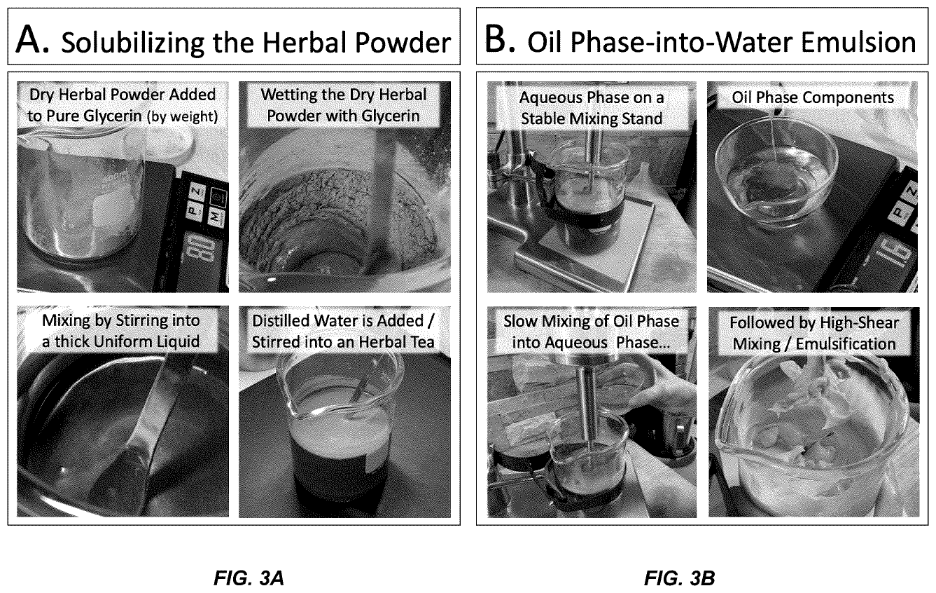

First, the Aqueous Phase was prepared. Powdered Andro-API (by weight (wt)) was wetted with pure Glycerin at a 3:1 ratio (Glycerin to Andro) and mixed into a uniform liquid consistency before further herb solubilization and hydration with distilled H2O (added at 76.0% by weight) to produce the Aqueous Phase (see FIG. 3A). Next, the Oil Phase was prepared. The specified amounts of Oil Phase components (as shown in Table 1, shown above) were weighed and mixed together, separately (see FIG. 3B).

The lesser Oil Phase (B) was then added to the larger Aqueous Phase (A) by simple mixing on a stable mixing platform (e.g. Dynamic MiniPro MX070 variable-speed mixer). Mixing and homogenization of A+B was accomplished at a slow speed (4-5 k RPM).

Mixing and Homogenization of the A+B was then followed by vigorous Emulsification—achieved by ramping-up the speed of a Dynamic MiniPro MX070 variable-speed mixer, in stages—best results were achieved using a 4-blade stirrer/impeller (˜3 k to 12 k RPM). At high speed (12 k RPM; ˜4-5) minutes, the uniform homogenate thickened and plumped into a semi-solid uniform emulsion, a light tan/brown gel-crème. Subsequently, the gel-crème was bottled into airless 10 ml and 50 ml plastic pump bottles (with ˜5% total loss).

### Example 2: Gel-Crème Formulation with Andrographolide for Pain and Inflammation of Spinal Arthritis

The gel-crème formulation was applied to the back of an elderly male subject for the treatment of pain and inflammation of spinal arthritis. The formulation was applied daily was applied topically, once daily, 5-7 ml (equivalent to 50-70 mg ANGL) over a 12 week period.

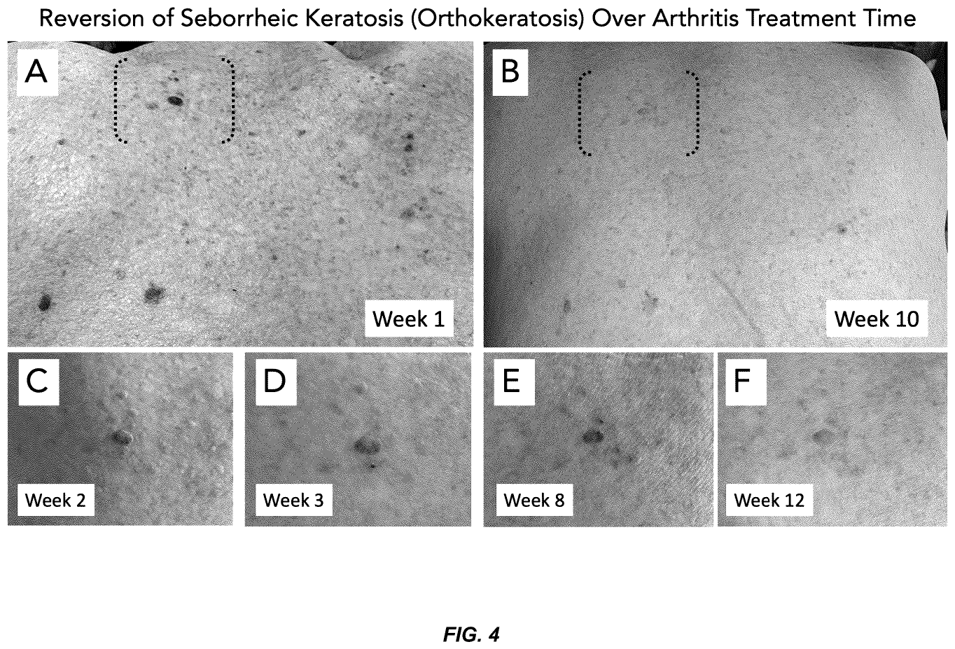

As shown in FIG. 4, panel A, at the start of administration, hyperplastic stem cell reversion to normalcy was observed in benign, yet possibly pre-cancerous, skin lesions on the back.

The formulation was clinically effective in the management of chronic spinal arthritis (pain and inflammation). The hyperplastic, melanotic, abnormal stem cells of Seborrheic Keratosis are seen here reverting toward normalcy by treatment week 10 FIG. 4, panel B. Further, as shown in FIG. 4 (panels B-F), improvements in skin health & quality were evident over a period of weeks. Long term treatment revealed a decrease in papillomatosis, hyperkeratosis, and hyperpigmentation: hyperplastic melanocytes and basoid cells (see, e.g., panel F) following daily applications of the Andrographis test gel-crème. The gel-crème formulation was applied to the back of the elderly male subject to assess the prospective treatment efficacy on spinal arthritis.

The observed dermal reversion of hyperplasia upon topical application, is evidence of potent ANDRO bioactivity on skin and dermal tissues (see FIG. 4); however, the appreciable transdermal delivery to deeper muscular tissues and presumably to the underlying lymphatics and vasculature, in the following minutes after topical application, represents the potential for delivering Andrographolide diterpenes systemically, while avoiding oral inefficiencies and first-pass metabolism. Note: the recommended medical/oral doses of Andrographolides for adults is: 50 to 60 mg ANGL, standard dosing; 80 mg ANGL extra strength oral doses; and 100 mg represents maximum recommended oral doses.

### Example 3: Creation and Use of a Gel-Crème Formulation with EGCG and ALA

Using the processes outlined in Example 1 above, a gel-crème formulation with EGCG and ALA (an EGCG-protective co-antioxidant) was prepared for testing. Table 2 below outlines the components and amounts of each ingredient used in the composition.

TABLE 2

| | | |
|---|---|---|
| Reagents/Phase | % Formula | 400 g (total wt) |

A: Aqueous Phase

| Pure Distilled Water | 76.7% | 308 | g |
|---|---|---|---|
| Pure Glycerin | 7.0% | 28 | g |
| Pure EGCG (94%) | 1.0% | 4.0 | g |
| Alpha Lipoic Acid | 0.6% | 1.2 | g |
| Total (Aqueous Phase) | 85.3% | 341.2 | g |
| B: Oil Phase | | | |
| Caprylic/C. Triglycerides | 4.0% | 16 | g |
| Refined Avocado Oil | 3.0% | 12 | g |
| Dimethicone 350 | 3.0% | 12 | g |
| SepilifeNude, Copolymer | 3.0% | 12 | g |
| (Emulsifier/Rheol. Blend) | | | |
| Frag. Oil: Tea/Cucumber | 0.2% | 0.8 | g |
| Tocopherol (Vitamin E) | 0.5% | 2 | g |
| Euxyl PE 9010 | 1.0% | 4 | g |
| Total (Oil Phase) | 14.7% | 58.8 | g |
| Total: | 100.0% | 400 | g |

The EGCG/ALA gel-crème formulation was applied to the back/spine of an elderly male subject for the treatment of pain and inflammation of spinal arthritis with associated intervertebral disc disease (IVDD). For initial testing, approximately 4 mls of the test formulation was applied overnight—the subject reported a mild localized analgesia within minutes, followed by deep muscular relaxation and systemic effects ‘felt’ over a period of hours. Thereafter, approximately 3 to 5 mls of the formulation was applied twice per day for a period of 4 weeks, with positive effects on arthritis and IVDD reported (possible enhanced permeability and retention (EPR) effects), including spinal flexibility and localized pain control.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A topical transdermal formulation comprising:
a) Andrographolides (ANGL) (i.e., Andrographis paniculata, full spectrum extract), Epigallocatechin Gallate (EGCG), or a combination thereof;
b) a wetting/hydrating agent;
c) a vegetable oil;
d) a natural triglyceride emollient;
e) a polymeric emulsifier/rheological modifier;
f) a silicone oil;
g) a natural antioxidant;
h) a lipid preservative; and
i) an antimicrobial/cosmetic preservative.

2. The topical formulation of claim 1, wherein the formulation comprises bioactive andrographolides, ANGL; and ANGL is a powdered extract of Andrographis paniculata (leaf) that is standardized to ≥50% ANGL.

3. The topical formulation of claim 2, wherein the ANGL is present in an amount of about 0.5% to about 5.0% based on the total weight of the formulation.

4.-6. (canceled)

7. The topical formulation of claim 1, wherein the formulation comprises a combination of EGCG and ANGL, wherein EGCG is a powdered extract of C. sinensis (leaf) that is ≥94% pure, and ANGL is a full spectrum extract that is >50% pure.

8. The topical formulation of claim 7, wherein the ANGL is present in an amount of about 0.5% to about 5.0% based on the total weight of the formulation.

9. (canceled)

10. The topical formulation of claim 1, wherein the formulation comprises a combination of EGCG and ALA, wherein is a powdered extract of C. sinensis (leaf) that is ≥94% pure.

11. The topical formulation of claim 10, wherein the EGCG is present in an amount of at least 1.0% or more based on the total weight of the formulation and the ALA is present in an amount of at least 0.6% or more based on the total weight of the formulation.

12. The topical formulation of claim 1, wherein the wetting/hydrating agent is glycerin, and wherein the wetting/hydrating agent further comprises distilled water.

13. (canceled)

14. The topical formulation of claim 12, wherein the glycerin is present in an amount of at least about 7.0% or more based on the total weight of the formulation.

15. The topical formulation of claim 1, wherein the vegetable oil is Avocado Oil, Macadamia Nut Oil, or Olive Oil, and is present in an amount of at least about 3.0% or more based on the total weight of the formulation.

16. (canceled)

17. The topical formulation of claim 1, wherein the natural triglyceride emollient is Caprylic/Capric triglyceride, and is present in an amount of at least about 4.0% or more based on the total weight of the formulation.

18. (canceled)

19. The topical formulation of claim 1, wherein the polymeric emulsifier/rheological modifier is Sodium Acrylate/Sodium Acryloyldimethyl Taurate Copolymer-C15-19 Alkane-Polyglyceryl-6 Laurate-Polyglycerin-6, and is present in an amount of at least about 3.0% or more based on the total weight of the formulation.

20. (canceled)

21. The topical formulation of claim 1, wherein the silicone oil reduces tackiness of the topical formulation.

22. The topical formulation of claim 21, wherein the silicone oil is Dimethicone DM350 (medium viscosity), and is present in an amount of at least about 3.0% or more based on the total weight of the formulation.

23. (canceled)

24. The topical formulation of claim 1, wherein the natural antioxidant and the lipid preservative is Tocopherol (Vitamin E), and is present in an amount of at least about 0.5% or more based on the total weight of the formulation.

25. (canceled)

26. The topical formulation of claim 1, wherein the antimicrobial/cosmetic preservative is Euxyl PE 9010, (phenoxyethanol+ethylhexylglycerin), and is present in an amount of at least about 1.0% or more based on the total weight of the formulation.

27. (canceled)

28. The topical formulation of claim 1, further comprising a fragrance.

29. The topical formulation of claim 22, wherein the fragrance is obtained from an essential oil.

30. The topical formulation of claim 29, wherein the essential oil is roasted coconut or green tea and cucumber.

31.-32. (canceled)

33. The topical formulation of claim 1, wherein the formulation is formulated as a broad-spectrum anti-viral agent/adjunct.

34. The topical formulation of claim 1, wherein the formulation is formulated as a broad-spectrum anti-cancer agent.

35. A method of treating Arthritis and Rheumatoid Arthritis; Muscle Ache (Polymyalgia Rheumatica); Peripheral Neuropathies; Diabetic Neuropathies; CNS & Aging Dementias; Multiple Sclerosis; or Palpable cancers, comprising administering to a subject in need thereof the topical formulation of claim 1.

36. A method of treating a cancer, comprising administering to a subject in need thereof: a cancer chemotherapy or a cancer gene therapy, and the topical formulation of claim 1.

37. A method of treating a disease or disorder, comprising administering to a subject in need thereof: a cell-based therapy, and the topical formulation of claim 1.
